(19) United States
(12) Patent Application Publication
(10) Pub. No.: US 2007/0179110 A1
(43) Pub. Date: Aug. 2, 2007

(54) EXPRESSION OF IMMUNOGENIC SUBSTANCES

(75) Inventors: Stephen James Russell, Rochester, MN (US); Luis Alvarez-Vallina, Madrid (ES); Siamak Agha-Mohammadi, Pittsburg, PA (US); Robert Hawkins, Cheshire (GB)

Correspondence Address: FISH & RICHARDSON P.C., PO BOX 1022, MINNEAPOLIS, MN, 55440-1022, US

(73) Assignee: Mayo Foundation for Medical Education and Research, Rochester, MN (US)

(21) Appl. No.: 11/611,708

(22) Filed: Dec. 15, 2006

Related U.S. Application Data
(63) Continuation of application No. 10/756,818, filed on Jan. 13, 2004, now abandoned.
Application No. 10/756,818 is a continuation of application No. 09/197,056, filed on Nov. 20, 1998, now abandoned.
Application No. 09/197,056 is a continuation-in-part of application No. PCT/GB98/02693, filed on Sep. 7, 1998.
(60) Provisional application No. 60/083,657, filed on Apr. 30, 1998.
(60) Provisional application No. 60/076,448, filed on Mar. 2, 1998.

(30) Foreign Application Priority Data
Nov. 7, 1997 (GB) 9723448.8
Sep. 6, 1997 (GB) 9718872.6

(51) Int. Cl. A61K 48/00; C12N 15/09

(52) U.S. Cl. 514/44; 435/455

(57) ABSTRACT

Disclosed is a method of regulating the expression in a human or animal subject of a nucleic acid sequence encoding a polypeptide which is immunogenic in the subject; the method comprising introducing into a mammal a cell comprising the nucleic acid sequence encoding the immunogenic polypeptide, said sequence being operably linked to a drug-regulatable promoter; and altering the concentration of regulatory drug to which the cell is exposed.

### BACKGROUND OF THE INVENTION

The invention relates to methods of altering the sensitivity of a leukocyte to a target antigen.

Unlike antibody molecules, T cells can migrate actively and efficiently through microvascular walls (a process known as extravasation), allowing them to penetrate the core of a solid tumor before they exert their cytolytic function. Ex vivo expansion and re-infusion of autologous tumor-reactive T cells is being explored as an experimental approach to cancer therapy. However, circulating T cells from peripheral blood lack specificity for tumor antigens (1) and it is often impractical or impossible to obtain sufficient numbers of tumor infiltrating lymphocytes (2). To overcome these problems new approaches have been developed whereby antibody specificity can be combined with the efficient trafficking properties and effector functions of T cells. Several reports have demonstrated the feasibility of transfecting cultured T cells with genes encoding chimeric receptors in which single-chain antibody domains (scFv) are linked to different signalling portions of the TCR/CD3/ζ complex as a means to target T cells towards native antigens (3-6). However it is apparent that the long-term clinical success of this “T-body” approach could depend on the development of solutions to a number of problems.

Perhaps the most significant concern is that since few “Cancer antigens” are truly tumor-specific (7), successful therapy with tumor-reactive T-bodies could be associated with significant collateral damage to normal tissues expressing low levels of the targeted antigen. It will therefore be desirable to develop strategies by which T cells can be rendered temporarily or permanently anergic to their target antigen, or differentially sensitive to different surface densities of the antigen on target cell membranes.

Although different strategies have been developed for regulating transgene expression in eukaryotic cells (8), the tetracycline-regulatable system (TRS) avoids the problems related to many of these systems by offering substantial regulation of transgene expression in response to concentrations of tetracycline that cause little or no toxicity in mammalian cells (9, 10). Miller & Whelan (1997 Hum. Gene Therapy 8, 803-815) have recently reviewed progress towards the development of regulatable vectors for gene therapy. Among the vectors described are those using the TRS.

In the TRS originally described by Gossen & Bujard (9), the tetracycline repressor protein was fused to the Herpes Simplex Virus (HSV) VP16-activating domain, to create a chimeric tetracycline-repressible transactivating (tTA) polypeptide, which binds to DNA comprising the tet operator sequence, causing transcriptional activation of coding sequences downstream of the operator. The presence of tetracycline or analogues thereof (such as doxycycline, anhydrotetracycline, minocycline and oxytetracycline) inhibits this transcriptional activation, as these compounds bind to the tTA polypeptide, altering its conformation and prevent its binding to the tet operator sequence.

Variants of the original TRS have now been described (WO 96/01313) in which a mutant form of the tet repressor protein binds to DNA in the presence, but not in the absence, of tetracycline or its analogues. Thus, in these systems, expression of a tet operator-linked gene is positively regulated in the presence of tetracycline or its analogues.

One object of this invention is to produce circulating leukocytes that elicit an anti-tumor effect due to their regulated expression of an immunogenic polypeptide and a cell surface component that targets them to the tumor cells. Anti-tumor effect means capable of decreasing the size of a tumor by at least 10%, preferably by 20-50% and most preferably, by 100%. Preferably, the decrease in the size of a tumor is permanent.

Another object of the invention is to produce leukocytes that are specifically tumor reactive because they respond to a target antigen present on the surface of both normal and tumor cells in a density dependent manner.

### SUMMARY OF THE INVENTION

The invention features methods of producing circulating T cells from peripheral blood that are targeted to and react against tumor cells. The invention also features methods for producing circulating T cells from peripheral blood with specificity for tumor antigens. Tumor antigens are not always tumor specific and are often expressed at low levels on normal tissues. Included in this invention are methods of regulating the expression of immunogenic polypeptides and altering the sensitivity of a leukocyte to a target antigen thereby producing T cells that are specifically tumor reactive.

The invention provides a method of regulating the expression of a nucleic acid sequence encoding a polypeptide which is immunogenic in a mammal. As used herein, “mammal” refers to animal or human. The method includes the steps of introducing into a mammal a cell comprising the nucleic acid sequence encoding the immunogenic polypeptide, wherein the sequence is operably linked to a drug-regulatable promoter; and altering the concentration of regulatory drug to which the cell is exposed.

In a preferred embodiment of this method, the cell is a leukocyte.

In other preferred embodiments the cell is a lymphocyte (B or T lymphocytes), monocyte or macrophage.

In other preferred embodiments, the mammal has already made an immune response to the immunogenic polypeptide.

In other preferred embodiments, the mammal has circulating antibodies which react with the immunogenic polypeptide.

In other preferred embodiments, the mammal has immunocompetent memory cells specific for the immunogenic polypeptide.

In other preferred embodiments, prior to introduction of the cell into the mammal the expression of the immunogenic polypeptide is substantially inhibited in vitro; and wherein expression of the immunogenic polypeptide reaches a maximum level in the mammal after a delay interval.

In other preferred embodiments, expression of the immunogenic polypeptide is inhibited in vitro by exposure of the cell to the regulatory drug, and wherein expression in the mammal is induced after a delay interval, the mammal is substantially free of the regulatory drug.

In other preferred embodiments, expression of the immunogenic polypeptide is inhibited in vitro by substantial absence of the regulatory drug; and wherein expression in the mammal is induced after a delay interval by administration to the mammal of the regulatory drug. Substantial absence means an amount that is undetectable by immunological or enzymatic methods of detection. In particular, the substantial absence of a regulatory drug refers to an amount or regulatory drug that does not stimulate an increase or decrease in the expression of an immunogenic polypeptide sequence that is operably linked to a promoter that is regulated by this same regulatory drug.

In other preferred embodiments, the regulatory drug is selected from the group consisting of: tetracycline or an analogue thereof (herein defined); glucocorticoid steroids; sex hormone steroids, lipopolysaccharide (LPS); and isopropylthiogalactoside (IPTG).

In other preferred embodiments, the immunogenic polypeptide exerts a therapeutic effect in the mammal.

In other preferred embodiments, the immunogenic polypeptide exerts an anti-tumor effect in the mammal.

In other preferred embodiments, the nucleic acid sequence encodes a replicable viral genome or a viral vector.

In a related aspect, the invention also features a cell transformed with a nucleic acid sequence encoding a polypeptide which is immunogenic to a mammal. The nucleic acid sequence is operably linked to a drug-regulatable promoter, such that expression of the immunogenic polypeptide by the cell may be controlled by altering the concentration of regulatory drug to which the cell is exposed.

In a preferred embodiment of this method, the cell is a leukocyte.

In a related aspect, the invention also features a composition for use in a gene therapy method, comprising a plurality of a cell in accordance with the second aspect as defined above, in a physiologically acceptable diluent medium.

In a related aspect, the invention also features a method of making a composition for use in gene therapy. The method includes the steps of obtaining a sample of cells from a mammal; transforming the cells with a nucleic acid sequence encoding a heterologous immunogenic polypeptide, said nucleic acid coding sequence being operably-linked to a drug-regulatable promoter; selecting those cells successfully transformed; and mixing the selected cells with a physiologically acceptable diluent. Desirably, performance of the method of the fourth aspect defined above will result in the production of a composition suitable for use in the method of the first aspect of the invention.

Physiologically acceptable diluent means water, phosphate buffered saline, or saline, and further may include an adjuvant. Adjuvants such as incomplete Freund's adjuvant, aluminum phosphate, aluminum hydroxide, or alum are materials well known in the art.

Finally, the invention provides a method of regulating the expression of a nucleic acid sequence encoding a heterologous polypeptide in a leukocyte, comprising introducing into the leukocyte the nucleic acid coding sequence operably-linked to a tetracycline-operator sequence, and a sequence encoding a tetracycline-sensitive DNA-binding expression-regulating polypeptide; and altering the concentration of tetracycline (or analogues thereof) to which the leukocyte is exposed, so as to regulate expression of the coding sequence. The heterologous polypeptide may be any polypeptide not normally expressed in the leukocyte.

Altering the concentration means increasing or decreasing the amount. When culturing cells in the presence of steroids, the steroid concentration can be increased or decreased within the range of between 1 nM to 1 mM. When the steroid inducing agent is administered to a mammal, the dosage can be increased or decreased to preferably achieve a serum concentration between approximately 0.05 and 10.0 μg/kg. When culturing cells in the presence of IPTG the dosage is preferably increased or decreased within the range of approximately 1 μM to 100 mM and more preferably in the range of 100 μM to 1 mM. When culturing cells in the presence of LPS the dosage is preferably increased or decreased within the range of approximately 0.1 nM to 1 mM and more preferably in the range of approximately 100 μM to 1 mM. When culturing cells in the presence of tetracycline or a tetracycline analogue, the dosage is preferably increased or decreased within the range of between 10 to 1000 ng/ml. When the inducing agent is administered to a mammal, the dosage is preferably increased or decreased to preferably achieve a serum concentration between approximately 0.05 and 1.0 μl/ml.

Further features and advantages of the invention will become more fully apparent in the following description of the embodiments and drawings thereof, and from the claims.

### BRIEF DESCRIPTION OF DRAWINGS

FIGS. 1A and 1B are schematic representations of nucleic acid constructs referred to in the example below;

FIGS. 2A, 2B and 3 show representative FACS data;

FIG. 4 is a graph showing expression of a chimeric polypeptide (as a percentage of expression in control cells) against time; and

FIGS. 5A and 5B are bar charts showing the levels of IL-2 production (in picograms/ml) by T lymphocytes exposed to various concentrations of tetracycline or the tetracycline analogue, doxycycline.

### DESCRIPTION

The invention provides for methods of altering the sensitivity of a leukocyte to a target antigen by regulating the expression of immunogenic polypeptides in eukaryotic cells.

As used herein, “immunogenic polypeptide” refers to a protein that elicits a response from the immune system that has a therapeutic or anti-tumor effect.

As used herein, “drug-regulatable promoter” refers to portions of nucleic acid which regulate the expression of coding regions of nucleic acid, in response to the presence of a substance (the “regulatory drug”) exogenous to the cell.

As used herein, “regulatory drug” refers to a substance that modifies the activity of the drug regulatable promoter at a concentration which is non-lethal to the cell in which the nucleic acid is present.

As used herein, “transformation” refers to methods of introducing nucleic acid sequences into eukaryotic cells in vitro, including transfection, transduction, electroporation and cell fusion.

As used herein, “exogenous agent” refers to a substance which affects the expression of the leukocyte-activating molecule, preferably in a selective, specific manner.

The invention provides a method of regulating the expression of a nucleic-acid sequence encoding a polypeptide which is immunogenic. Preferably, the immunogenic polypeptide is a cytotoxic agent (e.g. an “immunotoxin”—that is, a toxic moiety fused to an immunoglobulin binding domain, or a targeting moiety having a specific binding activity), or an agent such as an immunoglobulin, antibody, bispecific antibody or any of the other known variants of antibodies (e.g. scFv), capable of recruiting tumour-infiltrating lymphocytes (TILs) into the tumor.

It is preferable to avoid expression of the immunogenic polypeptide until the introduced leukocyte has reached the tumor as it may cause: (a) collateral damage to non-malignant cells and/or (b) interaction of the immunogenic polypeptide with components of the mammal's immune system (especially circulating antibodies). This latter point is particularly pertinent if repeated administrations of the leukocytes are required, as this would cause efficient induction of immune responses to the immunogenic polypeptide, which would tend to interact with the administered leukocytes and prevent them from reaching the target tumor. These problems can be overcome by the method of the present invention, in which the therapeutic (immunogenic) polypeptide is only allowed to become expressed at high levels after a significant time delay, by which point the administered leukocytes will have penetrated the target tumor, thereby preventing interception by the immune system and minimising collateral damage to non-malignant cells.

The efficiency of a tumor attack by introduced leukocytes may be enhanced by causing the cells to express a cell-surface component which targets the leukocyte to a marker expressed on the surface of the tumor cells (see, for example, Eshar et al., 1993 Proc. Natl. Acad. Sci. USA 90, 720; Hwu et al., 1993 J. Exp. Med. 178, 361; Stancovski et al., 1993 J. Immunol. 151, 6577; and Brocker et al., 1996 Eur. J. Immunol. 26, 1770). These may be, for example, chimeric “T body” targeting components, known to those skilled in the art (see disclosures of Eshar et al., Hwu et al., Stancovski et al., and Brocker et al., cited above).

To delay expression of the immunogenic polypeptide following introduction of the cell expressing the polypeptide into the mammal, in a preferred embodiment, prior to introduction of the cell into the mammal the expression of the immunogenic polypeptide is substantially inhibited in vitro, and expression of the immunogenic polypeptide reaches a maximum level in the mammal after a delay. Substantially inhibited in vitro means being expressed at a level of less than 50% of the level of expression induced by the presence or absence of a regulatory drug, more preferably less than 10-25%, and most preferably less than 5%. Maximum level means the highest level of expression obtained due to the presence or absence of a regulatory drug. As used herein, the maximum level of expression cannot be increased any further upon the addition of an increased concentration of a regulatory drug. The invention is such that it takes a significant period, or a delay interval, (typically 2 to 10 days, preferably 4 days or longer) for the cell to move from the fully-repressed state to the fully-expressed state.

Many tumor-associated antigens are not unique to tumor cells: they are expressed at relatively high densities on the surface of tumor cells, but may also be expressed at lower density on the surface of certain non-tumor cell types in a patient. Accordingly, many therapeutic methods which attempt to target cytotoxic agents, or to direct immune responses, to tumor cells via a tumor associated antigen often result in collateral damage to non-tumor cells.

The present invention provides a method of targeting therapeutic effects specifically to cells expressing a high density of tumor associated antigen. Therapeutic effect means capable of ameliorating the symptoms or conditions, e.g. of a disease, by at least 10%, preferably by 20-50% and most preferably, by 100%. Expression of the leukocyte-activating molecule on the surface of the leukocyte (which delivers or mediates the therapeutic effect) can be regulated, thereby altering the density of leukocyte-stimulating molecule (e.g. tumor-associated antigen) needed to reach the threshold level of interaction at which the leukocyte becomes activated.

I. Regulatory Promoter Systems

A number of suitable drug-regulatable promoters and corresponding regulatory drugs are known (see for example, Miller & Whelan, Hum. Gene Ther. 8, 803-815), and include promoters regulated by tetracycline or an analogue thereof, glucocorticoid steroids, sex hormone steroids, lipopolysaccharide (LPS) and isopropylthiogalactoside (IPTG). As used herein, “tetracycline analogue” refers to compounds which are structurally related to tetracycline and which bind to the Tet repressor with a Kₐ of at least approximately 10⁶M⁻¹. Preferably, the tetracycline analogue binds with an affinity of about 10⁹M⁻¹ or greater. Examples of tetracycline analogues include but are not limited to doxycycline, anhydrotetracycline, minocycline, oxytetracycline, chlorotetracycline, epioxytetracycline and cyanotetracycline.

A. Properties

A number of properties are critical for effective control of gene expression by a regulatory promoter system. For effective gene regulation, the regulatory system must exhibit high transgene expression on induction and low basal expression on repression. Furthermore, the system must be regulated by a non-toxic inducible agent. The most efficient system for gene delivery involves a single vector containing both the regulatable DNA-binding expression-regulating polypeptide and the foreign gene under the control of this polypeptide. By using a single vector system, it is ensured that equal copy numbers of regulatable DNA-binding expression-regulating polypeptide and reporter gene units will be integrated at the same chromosomal locus in a direct cis configuration.

Regulatory promoter systems often utilize drug-regulatable promoters. The drug-regulatable promoter may be directly drug-regulatable, or more typically, indirectly drug-regulatable. An example of a system comprising an indirectly drug-regulatable promoter is a system in which a drug-regulatable operator exerts a regulatory effect (that is dependent on the concentration of regulatory drug) on a promoter. Regulatable promoter systems can be modified to respond either positively or negatively to the drug.

It may be desirable to modify the components of the drug-regulatable promoter system employed in the method so as to reduce their immunogenicity (e.g. by modifying any DNA-binding protein so as to remove certain epitopes). Conveniently, if present, the DNA-binding protein will comprise a nuclear localization signal (NLS), so as to minimise the amount which might become presented to the mammal's immune system. Other useful techniques for altering the components of the drug-regulatable system so that they do not elicit an immune response in the host cell are disclosed in WO 96/01313.

B. Regulatory Promoter Systems

A number of regulatory promoter systems can be used to express transgenes according to the methods described in this invention. Suitable drug-regulatable promoters include the tetracycline promoter, and glucocorticoid steroids, sex hormone steroids, lipopolysaccharide (LPS) and isopropylthiogalactoside (IPTG) regulatable promoters. However, these control systems can be problematic because their inducing agent(s) can be toxic or have pleiotropic effects. In the case of RNA polymerase II promoters, induction may involve factors that modulate the activity of numerous endogenous promoters. Furthermore, the basal level of activity may be high, in part, because endogenous activators interfere with the regulation of activity.

Yeast transcriptional regulatory systems involving the DNA-binding proteins GAL-4 and LEU3 are also functional in mammalian cells.

C. The Tetracycline-Regulatable System

To avoid potential, pleiotropic effects of the exogenous agent, a regulatable expression system should involve a transcriptional regulator that binds non-mammalian DNA motifs in response to a synthetic compound, and a regulatory sequence that is responsive only to this transcriptional regulator. Certain prokaryotic transcriptional systems have been modified to meet these requirements.

The TRS, which is founded on the efficiency of the tetracycline resistance operon of E. Coli, is a preferred regulatory promoter system. Very low concentrations of tetracycline are required for efficient gene repression. The binding constant between tetracycline and the tet repressor is high while the toxicity of tetracycline is low, thereby allowing for regulation of the system by tetracycline concentrations that do not affect cellular growth rates or morphology. Binding of the tet repressor to the operator occurs with high specificity. The pharmacological properties of tetracycline are well defined and favourable (rapid uptake into cells and penetration of the placenta and the blood/brain barrier).

The TRS is particularly useful for the method of the present invention which specifies that in some embodiments the presence of tetracycline will inhibit expression of the tetracycline operator (tet O)-linked coding sequence (i.e. the sequence which encodes the immunogenic polypeptide), while in other embodiments the presence of tetracycline will enhance expression of the tet O-linked sequence. Depending on the nature of the tet operator DNA binding protein, gene expression is either positively or negatively regulated in the presence of tetracycline. In the absence of tetracycline the wild-type bacterial tet repressor protein causes negative regulation of bacterial tetracycline-resistance genes: tetracycline binds to the repressor protein and prevents it from binding to the tet operator DNA sequence, thus allowing expression of the resistance genes. Conversely, a chimeric polypeptide comprising part of the tet repressor and the HSV VP16 transactivating domain causes positive regulation (transcriptional activation) of coding sequences. Other transactivating domains are known to those skilled in the art (e.g. amino acid residues 753-881 of GAL4; amino acid residues 399-499 of CTF/NF1; and those from ITF1 or ITF2), and may be used, instead of VP16, to form a chimeric tetracycline-sensitive polypeptide.

In addition to the chimeric polypeptides described in the prior art (e.g. Gossen & Bujard 1992, cited above) which comprise a portion of the wild type tet repressor protein, other polypeptides (disclosed in WO 96/01313) which comprise a mutated form of the tet repressor protein and which bind to the tet operator sequence in the presence, but not in the absence, of tetracycline are known. When the TRS is used in the presence of the wild-type tet repressor, gene expression occurs in the absence of tetracycline. It may, therefore, be difficult to rapidly remove tetracycline and rapidly induce gene expression. In contrast, when this system is used with the mutant tet repressor the addition of tetracycline rapidly induces gene expression.

The tet operator (tet O) sequence is now well-known to those skilled in the art. For a review, the reader is referred to Hillen & Wissmann (1989) in Protein-Nucleic Acid Interaction. “Topics in Molecular and Structural Biology”, eds. Saenger & Heinemann, (Macmillan, London), Vol. 10, pp 143-162. Typically the nucleic acid sequence encoding the immunogenic polypeptide will be placed downstream of a plurality of tet O sequences: generally 5 to 10 such tet O sequences are used, in direct repeats.

Conveniently, the tet O sequences will be fused substantially adjacent (i.e. within 100 bp, preferably within 50 bp) to a “minimal” (i.e. enhancerless) eukaryotic promoter (such as the minimal CMV immediate early promoter, described previously [Gossen & Bujard 1992 Proc. Natl. Acad. Sci. USA 89, 5547]), such that binding of a transactivating tetracycline-sensitive polypeptide to the tet O sequence will cause enhanced expression of the tet O-linked coding sequence.

Constructs particularly suitable for introduction of the sequence encoding the immunogenic polypeptide and/or the drug-regulatable promoter are well-known and have been disclosed previously (e.g. Baron et al., 1995 Nucl. Acids Res. 23, 3605-3606; Schultze et al., 1996 Nature Biotechnology 14, 499-503). Particularly preferred constructs (where tetracycline, or a tetracycline analogue, is the regulatory drug), which allow for very high levels of regulation of expression of tet O-linked sequences, are pSiaII and pSiaIV.

pSiaII is a single vector that contains both the tetracycline-controlled transactivator (tTA) and the tTA-responsive promoter (tRP) transcribing mouse GM-CSF. This vector shows a 15-fold gene regulation in HeLa cells. pSiaII can be modified such that mouse GM-CSF can be replaced with any gene of interest, wherein the gene of interest is preferably a therapeutic gene, according to the invention.

pSiaIV is an enhancerless positive feedback regulatory vector transcribing both tTA and mGM-CSF from a modified tTA-responsive bidirectional promoter. pSiaIV can be modified such that mouse GM-CSF can be replaced with any gene of interest, wherein the gene of interest is preferably a therapeutic gene, according to the invention. This positive feedback bidirectional promoter system allows for high maximal levels of expression, and low levels of tetracycline repressed basal expression when expressed transiently or when stably integrated into a variety of cell lines. Furthermore, pSiaIV has a number of advantages over previously described TRS. This vector allows for regulation of both the activity and level of expression of the tetracycline-sensitive DNA-binding expression-regulating polypeptide. As a result of this dual regulation, the amount and activity of the tetracycline-sensitive DNA-binding expression-regulating polypeptide can be kept sufficiently low. Consequently, stable clones expressing this regulatory polypeptide will survive because there are no cytotoxic effects caused by overproduction of this protein. This improved methodology for downregulating the amount and activity of the tetracycline-sensitive DNA-binding expression-regulating polypeptide will also facilitate the production of cells expressing a tet O-linked sequence encoding a toxic protein. pSiaIV allows for 200-fold gene regulation in HeLa cells.

Construction of pSiaII and pSiaIV

The plasmid pUHD 15-1 contains the tTA transactivator gene transcribed from the CMV IE promoter, pUHD 10-3 carries a tRP followed by a polylinker, and pUTHC 13-3 contains a luciferase reporter gene under control of tRP (9). The 462 base pair XbaI/BamH I digested fragment from pCEP4. mGM-CSF containing mGM-CSF gene, was blunt ended with klenow (Cambio, Cambridge, UK) and inserted into the Xba I site of pUHD 10-3 following the treatment of this site with klenow and calf intestine alkaline phosphatase (CIP, Boehringer Mannheim, East Sussex, UK) to construct pCMV*⁻¹mGM-CSF.

pCMVmGM-CSF was constructed by digesting pCMV*⁻¹mGM-CSF with NheI, blunt-ending this site, and cutting the plasmid again with BamHI. This fragment was inserted into pUHD 15-1, cut with EcoRI, blunt-ended and digested again with BamHI. To construct pCEP4.tTA, the hygromycin expression cassette together with the chloramphenicol resistance gene, OriP, and the EBNA1 gene of pCEP4.Cm^{R} were removed by digestion with ClaI and NruI. This fragment was blunt-ended with klenow and inserted into the XhoI site of pUHD 15-1 following the treatment of this site with klenow and calf intestine alkaline phosphatase. pCEP4.Cm^{R} is an EBV based vector whereby the CMV promoter and SV40 poly(A) sequences of parental pCEP4 (Invitrogen, R&D Systems, Abingdon, UK) are replaced with the chloramphenicol resistance gene from pACYC184 (New England Biolabs, Inc.).

For construction of pSiaII, pUHD 15-1 was cut with Bam HI, blunt-ended, and digested again with EcoRI to release the 1 Kb fragment containing the tTA coding sequence. This was inserted into pCI.neo (plasmid containing chimeric intron, Promega, Southhampton, UK) digested at Not I site, blunt-ended and cut again with EcoRI to construct pCI.tTA.neo. The 1.35 Kb mGM-CSF expression cassette was removed from pCMV*⁻¹mGM-CSF by partial digestion with PvuII and complete digestion with Ssp I. This fragment was inserted into the unique Bam HI site of pCI.tTA.neo following the treatment of this site with klenow and CIP. Plasmids containing the mGM-CSF expression cassette in the opposite direction to the tTA and neomycin phosphotransferase expression cassettes (being in sense) were selected.

For construction of pCMV*⁻¹tTA/mGM-CSF, the tTA sequence was removed from pUHD 15-1 plasmid with EcoRI and BamHI digestion and inserted into pUHD 10-3 cut with the same restriction enzymes to construct pCMV*⁻¹tTA. The resulting plasmid was cut with PvuII and partially with SmaI. The 1.8 Kb tTA expression cassette was inserted at blunt-ended, CIP treated XhoI site of the pCMV*⁻¹mGM-CSF plasmid in opposite direction to the expression of mGM-CSF. The plasmid pCT*⁻¹tTA/mGMCSF was constructed by removing the tTA expression cassette from pCI.tTA.neo with SgfI and DraIII. This fragment includes the CCAAT and TATA boxes of the CMV IE promoter, the chimeric intron, the tTA sequence, and the SV40 poly (A). It was ligated into the blunt-ended, CIP treated XhoI site of pCMV*⁻¹mGM-CSF plasmid in opposite direction to the expression of mGM-CSF.

To construct pSiaIV, the oligonucleotides 5′TCGAGGCGGCCGCACCATGT (SEQ ID No.:3) 3′ and 5′ CTAGACATGGTGCGGCCGCC 3′ (SEQ ID No.:4) (translational initiation codon underlined) were annealed and ligated between the Xho I and Xba I sites to replace the translational initiation sequence of tTA. This will replace the immediate upstream sequence to translational initiation codon by CGCACC.

Suitable constructs for achieving the embodiment of the invention whereby the TRS controls the transcription of a replicable viral genome or a viral vector (comprising the sequence encoding the immunogenic polypeptide) have been disclosed by, inter alia, Hofmann et al., (1996) and Shockett et al., (1995) cited elsewhere. In preferred embodiments the replicable viral genome comprises substantially that of an adenovirus or a paramyxovirus (which genome may be artificially altered by conventional DNA manipulation techniques). Retroviral systems are particularly advantageous because they can be introduced (by infection) into a broad range of primary cells.

There are a number of difficulties and/or uncertainties relating to the use of the TRS. For example, Cooke et al., (1997 J. Gen. Virol. 78, 381-392) found that the TRS could not be used to regulate expression of the nef gene in the human T cell line Jurkat E6-1. Further, the HSV VP16 domain is associated with a toxic “squelching” effect, such that high levels of regulation have not previously been obtainable. A recent review (Miller & Whelan 1997 Hum. Gene Ther. 8, 803-815) has stated that the TRS “may not be applicable to all cell lines” (citing the work of Ackland-Berglund & Leib 1995 BioTechniques 18, 196-200; and Howe et al., 1995 J. Biol. Chem. 270, 14168-14174). However, as described above, these constructs overcome many of these difficulties.

II. Cells

The methods of this invention are suitable for leukocytes (T or B lymphocytes, monocytes or macrophages) because of their ability to migrate through blood vessel walls, penetrate a solid tumor, and, once within the tumor, exert a therapeutic effect (e.g. cytotoxic action, or recruitment of macrophages and lymphocytes). Any solid tumor, accessible by blood-bourne cells, could be available for treatment by the method of the invention.

Introduction of foreign cells into a mammal is likely to create an immune response against foreign antigens on the cell, so generally the cells will be tissue-matched with the recipient mammal. Most conveniently, the cells will be autologous cells originally obtained from the mammal (e.g. from peripheral blood, or from bone marrow), transformed in vitro with the relevant nucleic acid sequences, and then re-introduced into the mammal. Typically, the in vitro stages of the method will generally comprise a selection process to select those cells successfully transformed (as well as a selection for cells exhibiting a low basal level of expression of the tet O-linked sequence), and a growth stage, to increase the numbers of transformed cells. Methods of selection and growth of cells are well known to those skilled in the art and form no part of the present invention.

This invention includes methods for producing cells expressing a regulated amount of a leukocyte-activating molecule on their surface. The leukocyte-activating molecule can be a TCR or a chimeric TCR molecule. For example, the leukocyte may be transformed with a sequence directing the drug-regulatable expression of a TCR molecule, or of a chimeric TCR molecule which comprises at least the cytoplasmic signalling domain of the TCR molecule. Cytoplasmic or intracellular signalling domain means the region of a molecule that is located in the cellular cytoplasm and is capable of eliciting a response, for example increasing transcriptional activity, in the interior of a cell. Where the leukocyte-activating molecule is a chimeric TCR, the transmembrane domain (necessary to anchor the molecule on the surface of the T cell) may be from the wild type TCR molecule, or may comprise a transmembrane (TM) domain from any other molecule which is present on the surface of a eukaryotic cell. Preferably the TM domain is that from a member of the immunoglobulin family of polypeptides. Transmembrane domain means the hydrophobic region of a molecule that passes through a cell membrane and interacts with the hydrophobic tails of the lipid molecules in the interior of the membrane bilayer. The transmembrane domain is required to anchor the molecule to the surface of a cell.

The extracellular domain of the chimeric TCR molecule typically comprises a domain which has binding specificity for the leukocyte-stimulating molecule. Extracellular domain means the region of a molecule located on the exterior side (e.g. the non-cytoplasmic side) of the cell membrane. In preferred embodiments the specific binding domain is from an antibody or an antigen-binding fragment thereof (such as an scFv, Fab or the like). T lymphocytes can be transformed with a nucleic acid sequence directing the tetracycline-sensitive expression of a chimeric TCR molecule having specific binding activity for a tumor associated antigen. Adjustment of the amount of tetracycline (or analog thereof) administered to the patient can be made, such that the density of tumor-associated antigen on tumor cells is sufficient to cause activation of the T cell, while the density of the tumor-associated antigen on non-tumor cells is too low, minimising collateral damage. The desired adjustment of tetracycline concentration in the patient can be made by analysis of clinical symptoms or markers. The additional drug-regulatable promoter systems described above might also be used to regulate the expression of the leukocyte-activating molecule.

III. Drugs

Preferably the action of the regulatory drug is substantially specific for the drug-regulatable promoter such that, if it is necessary to administer the drug to the patient it will not cause widespread effects on other tissues or cell types. Such specificity is most readily conferred by use of a synthetic regulatory drug and a corresponding drug-regulatable promoter which is not normally present in the mammal.

Preferably the exogenous agent is one which can be administered to a human patient in a pharmacological manner: that is, the agent exerts an appropriate effect on the expression of the leukocyte-activating molecule at a concentration lower than that which causes any significant toxic effect on the patient.

As stated above, regulatory drugs should be able to either positively or negatively regulate a promoter. Generally it is preferred that the presence of regulatory drug inhibits expression of the immunogenic polypeptide, as subsequent removal of the cell from regulatory drug exposure normally gives a longer lag phase or delay before induction of expression of the immunogenic polypeptide.

Those skilled in the art will appreciate that a number of analogues of tetracycline are known, which can readily be substituted for tetracycline in the method of the invention. Preferred analogues are doxycycline and anhydrotetracycline. Other analogues include minocycline, oxytetracycline, chlorotetracycline, epioxytetracycline and cyanotetracycline. Other analogues of tetracycline are described by Hlavka & Boothe (“The Tetracyclines” in “Handbook of Experimental Pharmacology 78, Blackwood et al., (eds.) Springer Verlag 1985).

Certain analogues may actually be preferred to tetracycline, as they may have higher binding affinities (for the tetracycline-sensitive polypeptide) and so exert a regulatory effect at lower concentrations. Tetracycline analogues are not identical in their ability to induce gene expression and, based on the desired level of induction, a particular tetracycline analogue can be used. By using different tetracycline analogues in the TRS, the level of expression of a tet O-linked sequence can be modulated. The choice of a particular tetracycline analogue should also be based on the time course for resumption of activity of the tetracycline regulated promoter following removal of the particular analogue.

Drugs modulating other promoter regulatory systems include but are not limited to glucorticoid steroids, sex hormone steroids, lipopolysaccharide (LPS) and isopropylthiogalactoside (IPTG). When culturing cells in the presence of steroids, a preferred concentration range for the inducing agent is between 1 nM to 1 mM. When the steroid inducing agent is administered to a mammal, the dosage is adjusted to preferably achieve a serum concentration between approximately 0.05 and 10.0 μg/kg. When culturing cells in the presence of IPTG the dosage is preferably in the range of approximately 1 μM to 100 mM and more preferably in the range of 100 μM to 1 mM. When culturing cells in the presence of LPS the dosage is preferably in the range of approximately 0.1 nM to 1 mM and more preferably in the range of approximately 100 μM to 1 mM.

A therapeutically effective regimen may be sufficient to arrest or otherwise ameliorate symptoms of a disease. An effective dosage regimen requires providing the regulatory drug over a period of time to achieve noticeable therapeutic effects wherein symptoms are reduced to a clinically acceptable standard or ameliorated. The symptoms are specific for the disease in question. For example, when the disease is associated with tumor formation, the claimed invention is successful when tumor growth is arrested, or tumor mass is decreased by at least 50% and preferably 75%.

IV. Dosage and Administration

Methods of introducing the transformed cell into the mammal are well known. Conveniently this is done by infusion or injection into the mammal's bloodstream. Preferably 10⁵-10⁸ cells and more preferably 10⁶-10⁷ will be infused into the mammal's bloodstream. Methods of administration of the regulatory drug include oral administration (dissolving the inducing agent in the drinking water), slow release pellets and implantation of a diffusion pump. When culturing cells in the presence of tetracycline or a tetracycline analogue, a preferred concentration range for the inducing agent is between 10 to 1000 ng/ml. When the inducing agent is administered to a mammal, the dosage is adjusted to preferably achieve a serum concentration between approximately 0.05 and 1.0 μg/ml.

V. Therapeutic Genes

Although the method of the invention may be useful in regulating the expression of any polypeptide immunogenic to the mammal in question, it will be particularly useful for regulating the expression of one or more therapeutic polypeptides, especially in a mammal. Those skilled in the art are familiar with the wide range of polypeptides which may have potential usefulness when expressed as therapeutic polypeptides in patients by means of gene therapy techniques, but which may present difficulties because of their immunogenicity when expressed in a human patient because they are not normal human proteins.

One category of therapeutic polypeptides are polypeptides that are immunogenic due to the fact that they are not normal human proteins. Examples of such immunogenic polypeptides include proteins from other sources (e.g. from plants, animals, fungi, bacteria, yeasts and the like), and chimeric polypeptides which comprise portions of proteins from non-human sources, or even chimeric polypeptides which create novel fusions of human proteins or parts thereof and which are therefore immunogenic in a mammal.

Another category of therapeutic proteins are those which are not expressed or are not expressed in a functional form due to a genetic defect. Examples of such polypeptides include growth factors and blood clotting factors, hormones, neurotransmitters, enzymes, apolipoproteins, receptors, drugs, oncogenes, tumor antigens, tumor suppressors, structural proteins, viral antigens, parasitic antigens and bacterial antigens.

Specific examples of these compounds include CFTR, insulin, adenosine deaminase, proinsulin, growth hormone, dystrophin, androgen receptors, insulin-like growth factor I, insulin-like growth factor II, insulin-like growth factor binding proteins, epidermal growth factor TGF-α, TGF-β, PDGF, angiogenesis factors (acidic fibroblast growth factor, basic fibroblast growth factor and angiogenin), matrix proteins (Type IV collagen, Type VII collagen, laminin), phenylalanine hydroxylase, tyrosine hydroxylase, oncogenes (ras, fos, myc, erb, src, sis, jun), E6 or E7 transforming sequence, p53 protein, Rb gene product, cytokine receptor, Il-1, IL-6, IL-8, viral capsid protein, and proteins from viral, bacterial and parasitic organisms which can be used to induce an immunologic response, and other proteins of useful significance in the body. Any compounds for which there is an available nucleic acid sequence for the protein or polypeptide to be incorporated can be used according to the invention.

Another category of therapeutic agents useful according to the invention includes immunoactive agents, i.e., agents which combat viral infections or production by activating an immune response to the virus. Such agents include but are not limited to cytokines against viruses in general (Biron, 1994, Curr. Opin. Immunol. 6:530); soluble CD4 against SIV (Watanabe et al., 1991, Proc. Nat. Aca. Sci. 88:126); CD4-immunoglobulin fusions against HIV-1 and SIV (Langner et al., 1993, Arch. Virol. 130:157); CD4(81-92)-based peptide derivatives against HIV infection (Rausch et al., 1992, Biochem. Pharmacol. 43:1785); lympho-cytotoxic antibodies against HIV infection (Szabo et al., 1992, Acta. Virol. 38:392); IL-2 against HIV infection (Bell et al., 1992, Clin. Exp. Immunol. 90:6; and anti-T cell receptor antibodies against viruses in general (Newell et al., 1991, Ann. N.Y. Aca. Sci. 636:279).

The mammal is typically a human patient, but the method of the invention is potentially applicable to any mammal, such as domesticated mammals or farm animals. Accordingly, the method of the invention is typically performed in a mammal which has already made an immune response to the immunogenic polypeptide and who may have, for example, circulating antibodies which react with, or immunocompetent memory cells specific for the immunogenic polypeptide. Immunocompetent memory cells are cells of the immune system which have encountered an antigen, are not actively engaged in an immune response, but will readily become actively engaged in an immune response following a later encounter with the same antigen.

The invention will now be further described by way of illustrative example and with reference to the accompanying drawings.

### EXAMPLE 1

The inventors have evaluated the utility of the tetracycline-controlled transactivator system as a means to temporally regulate the expression of a surface molecule in a human T cell line. Using a vector containing both the transactivator and the expression gene unit, we were able to generate stably transfected Jurkat T cell lines in which the expression of a chimeric TCR (chTCR) molecule could be efficiently regulated. Depending on the tetracycline analogue used and its concentration, the induction of chTCR can be reversibly repressed to a greater or lesser extent. Furthermore, we have shown that fully repressed T cells can not be activated to produce IL-2 via this chimeric receptor, indicating that reversible functional inactivation of redirected T cells is possible.

The time-course to repress gene expression to basal levels was significantly shorter than the time-course for gene expression to reach maximal levels after drug removal, and the delay in resumption of promoter activity varied considerably depending on the concentration of doxycycline (a tetracycline analogue) used for repression. The relevance of these data to current ideas on T cell mediated immunotherapy is discussed.

Materials and Methods

Reagents.

The mAbs used included SPvT3b (mouse IgG2a) (11) and YTH913.12 (rat IgG2b) (Serotec Ltd., Oxford, UK) specific for human CD3E and CD28 molecules respectively. For direct staining the following FITC-conjugated antibodies were used: UCHT-1 (anti-CD3E, a mouse IgG1 Serotec; goat polyclonal antisera to mouse λ-light chain (Southern Biotechnology Associates, Inc, Birmingham Ala.); and goat polyclonal antisera to mouse IgG (γ-chain specific) (Sigma Chemical Co., St. Louis, Mo.). Bovine serum albumin (BSA) was conjugated with 4-hydroxy-5-iodo-3-nitrophenyl acetyl (NIP) (Cambridge Research Biochemicals, Northwich, UK) in a molar ratio of 10:1 (NIP₁₀-BSA) (12). Tetracycline hydrochloride (Sigma) was dissolved at a concentration of 0.5 mg/ml in culture medium. Doxycycline hydrochloride (Sigma) was dissolved in 0.02N HCl at a concentration of 1 mg/ml and further diluted in culture medium. The antibiotic solutions were freshly prepared on the day of use and diluted to the appropriate concentrations.

Vector Construction.

Plasmids pUHD 15-1, containing the tTA transactivator gene transcribed from the human CMV immediate early (CMV IE) promoter/enhancer, and pUHD 10-3, containing a tTA-responsive promoter (TRP, heptamerized tetO sequences (TetO)7 fused to a human CMV immediate early minimal promoter [PhCMV*−1]), were kindly provided by H. Bujard (9). Plasmid pCS was constructed by removing a 1308 bp Sal I fragment, containing the CMV IE promoter, the multiple cloning site and the SV40 polyadenylation signal, from pCEP4 backbone (Invitrogen, San Diego, Calif.). A 6723 bp Nru I-Cla I digested fragment from pCS was blunt ended with Klenow (Cambio, Cambridge, UK) and inserted into the Xho I site of plasmid pUHD 15-1 following the treatment of this site with Klenow and calf intestinal alkaline phosphatase (CIP, Boehringer Mannheim GmbH, Germany). The resulting plasmid containing both the tTA and the hygromycin transcription units in opposite directions was designated pCRAZY.

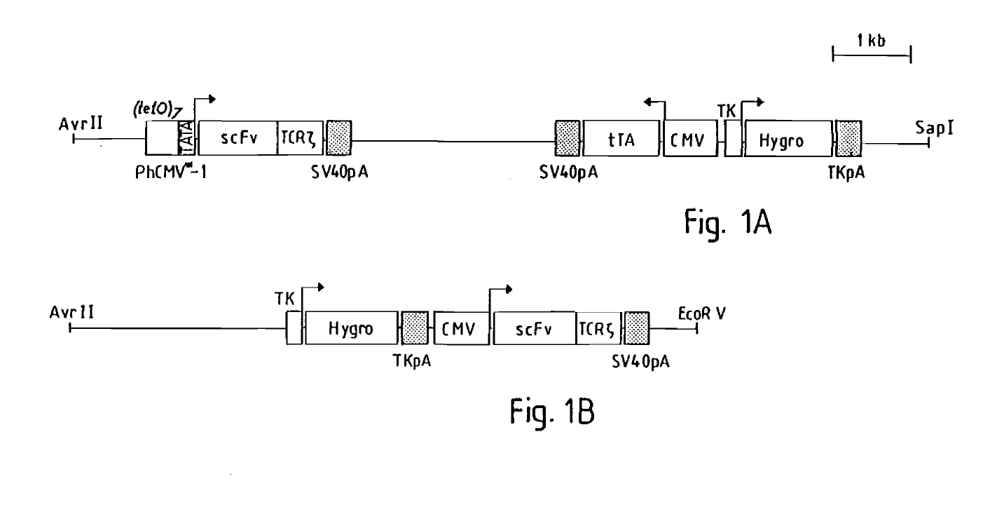

A chimeric NIP-specific scFv-TCR ζ molecule was constructed as described previously (12) and cloned into the plasmid pUHD 10-3. To do this the Hind III site from pUHD 10-3 was removed by cleavage with Hind III followed by Klenow fill-in and blunt-end ligation resulting in pLAV5. To construct pLAV6, a 1342 bp EcoR I-Xba I fragment derived from the plasmid pVACl.aNIP.TCR ζ (described in reference 12), containing a human VH1 leader sequence and a chimeric NIP-specific TCR ζ molecule, was cloned into the EcoR I-Xba I polylinker site of pLAV5. The 91 bp EcoR I-Hind III fragment containing a Rous Sarcoma virus (RSV) promoter partial sequence was removed from pLAV6 by digestion with EcoRI and Hind III, Klenow fill-in and blunt end ligation to produce plasmid pLAV7. A polylinker containing restriction sites unique to the vector construct, HindIII-Bgl II-EcoRV-Cla I, (5826: 5′-CATCGATCGAACTGATATCAGCAGATCTCAGAAGCTTAAT-3′ (SEQ ID No.:1) and 5827: 5′-ATTAAGCTTCTGAGATCTGCTGATATCAGTTCGATCGATGACGT-3′) (SEQ ID No.:2) was ligated into the Ssp I-AatII site of pLAV7 resulting in pLAV8. To construct a single plasmid with both the tTA transactivator gene and the chimeric NIP-specific scFv-TCR ζ gene under the control of TRP in antisense orientation (relative to the tTA transcription unit) the plasmid pCRAZY was digested with Xmn I and a Bgl II linker (New England Biolabs, Inc., Beverly, Mass.) was introduced at this site. After digestion with Bgl II and Hind III a 9386 bp fragment was inserted into the Bgl II-Hind III site of pLAV8. The resulting plasmid was designated pLAV12 (FIG. 1A).

Cell Culture and Transfections.

The Jurkat T cell line (clone E6-l) was maintained in RPMI 1640 supplemented with 10% foetal calf serum (FCS), 2 mM L-glutamine, 100 U/ml penicillin, 100 μg/ml streptomycin and 25 mM HEPES buffer (all from GIBCO-BRL, Gaithersburg, Md.), referred to as complete medium (CM). To generate stable cell lines Jurkat cells were transfected with linearized plasmid DNA (10 μg) by electroporation, at 250 mV and 960 μF, as described previously (13). Transfectants were selected in CM supplemented with 0.4 mg/ml of hygromycin B (Calbiochem, San Diego, Calif.). Stable cell lines were established after 3-4 weeks and analyzed by FACS for expression of the chTCR. To select a population of chTCR ζ expressing cells, stable Jurkat cells transfected with the linearized pLAV12 or pCEP4.aNIP.TCR ζ derived plasmid fragments were FACS sorted (see below). The resultant populations were cloned twice by limiting dilution and screened for protein expression by flow cytometry.

Flow Cytometry and Cell Sorting.

Expression of cell surface proteins was performed by standard direct immunofluorescence as described (14) using saturating amounts of FITC-conjugated antibodies. Dead cells were excluded from analysis using a combination of propidium iodide and forward light scatter. Appropriate FITC isotype-matched irrelevant Abs were used in all experiments. The samples were analysed with a FACScan® (Becton Dickinson, Mountain View, Calif.). A minimum of 20,000 cells was analysed for each sample. Subsequent re-analysis of data was performed using the CELLQuest software (version 1.2) (Becton Dickinson). Additionally, cells stained with FITC-conjugated goat antisera to mouse λ light chain were sorted under sterile conditions on a cell sorter (FACScalibur, Becton Dickinson).

IL-2 Release Assay.

The cells were pre-incubated at a concentration of 5×10⁵ ml for 48 hours in CM in the absence or presence of tetracycline or doxycycline at the indicated concentrations. Subsequently the cells were washed, counted and stimulated (10⁵/well) in triplicate with plastic-immobilised NIP10-BSA conjugates (iNIP10-BSA) or plastic immobilised anti-CD3E mAb (ianti-CD3) in fresh CM alone or in the presence of the drugs (12). The plates were incubated at 37° C. in 5% CO₂. After 20 hours supernatants were harvested and assayed for IL-2 activity using an ELISA kit (Genzyme Diagnostics, Cambridge, Mass.).

Results

Design of the tet-Regulatable Vector.

To determine if the expression of a chTCR could be pharmacologically regulated in T cells, the inventors constructed the plasmids pLAV12 and pCEP4.aNIP.TCR ζ (FIG. 1). FIGS. 1A and 1B are schematic maps of representative plasmid fragments used in the experiments showing the predicted structure after integration into the host genome: (A) pLAV12 and (B) pCEP4.aNIP.TCRζ, respectively. The direction of transcription is indicated by arrows.

Both constructs encode a chimeric TCR molecule that has been described previously (12), and comprises the antigen combining site of the hapten-specific (NIP) mAb B1.8 (15) fused to the transmembrane and cytoplasmic regions of the human TCR ζ chain (16). pLAV12 (FIG. 1A) is a tetracycline-regulatable construct containing all the components of the TRS with the tTA gene under the control of the constitutive CMV IE promoter and the gene coding for the chTCR inserted under the control of the tTA-responsive promoter. In an attempt to reduce potential cis-regulatory enhancement of TRP activity, due to proximity to the other enhancer and promoter elements present on the vector, the tTA-response cassette was separated from the other transcriptional units by a 2500 bp fragment containing the pUC derived ColE1 origin of replication and the β-lactamase gene (FIG. 1A). In the control construct pCEP4.aNIP.TCRζ the chTCR molecule was under the control of the constitutive CMV IE promoter (FIG. 1B).

Both constructs encode a hygromycin resistance marker gene transcribed by a constitutive promoter. To promote the stable integration of these DNA constructs in the designed configuration in transfected T cells, a linearized Avr II-Sap I 9975 bp DNA fragment (FIG. 1A) from plasmid pLAV12 and a linearized Avr II-EcoRV 7551 bp fragment (FIG. 1B) derived from plasmid pCEP4.aNIP.TCRζ, both lacking the EBV replication origin and the EBNA-1 gene (FIG. 1), were used to transfect Jurkat cells.

tTA-Dependent Expression of the Chimeric scFv Gene Construct.

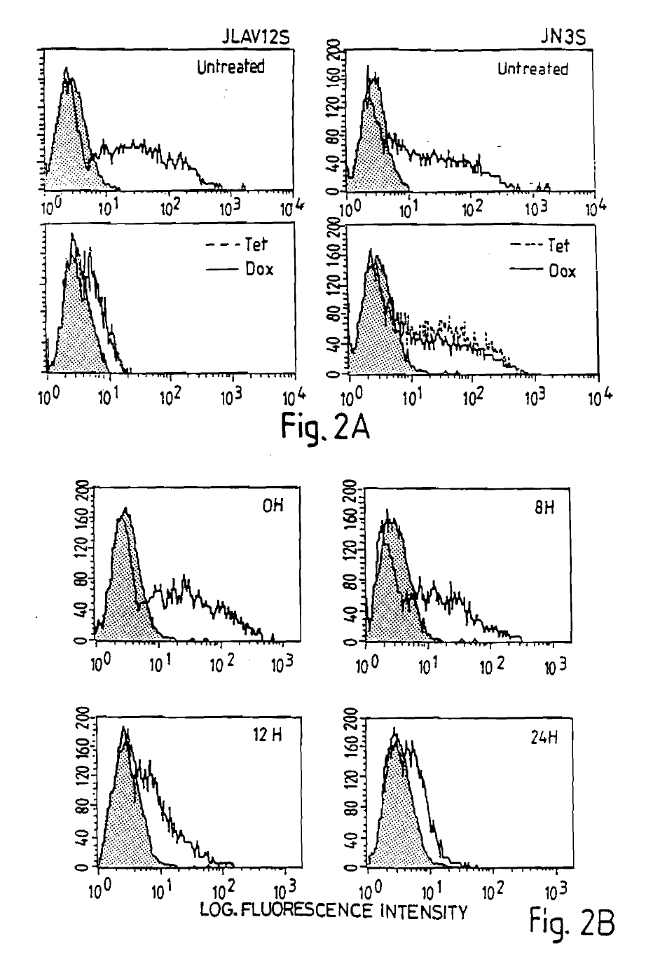

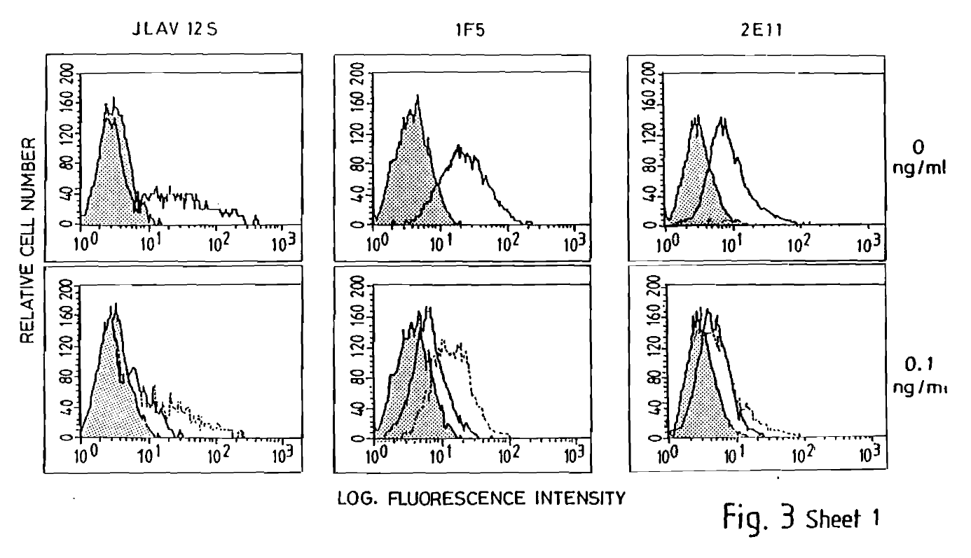

Jurkat E6-1 cells were transfected with linearized fragments of pLAV12 and pCEP4.aNIP.TCRζ. To select for higher expression of the chTCR, the stably transfected hygromycin-resistant cells were FACS sorted after staining with a FITC-labelled goat anti mouse λ-light chain antiserum. Most of the isolated pLAV12 transfectants (JLAV12S) and pCEP4.aNIP.TCRζ transfectants (JN3S cells), expressed the chTCR although there was considerable heterogeneity in the absolute levels of expression (FIG. 2A, 2B).

FIG. 2 shows representative results confirming the regulation of the chTCR gene expression by tetracycline analogues. In FIG. 2A, stable transfected uncloned JLAV12S (left hand side) and JN3S Jurkat (right hand side) cell populations were cultured for 48 hours in tetracycline-free medium (CM, upper row of panels) or in the presence of 1 μg/ml of Tet (broken line) or Dox (solid line) (lower row of panels) and the surface expression of chTCRs was examined after staining with FITC-conjugated goat antisera to mouse λ light chain. FIG. 2B shows a timecourse of inactivation of chTCR gene expression in JLAV12S cells zero hours (top left), 8 hours (top right), 12 hours (bottom left) or 24 hours (bottom right) after addition of Dox at 1 μg/ml. In both FIGS. 2A and 2B negative controls (FITC-conjugated goat antisera to mouse IgG) are overlaid (filled curve). The fluorescence channel number is plotted along the x axis, and the y axis represents the relative cell number.

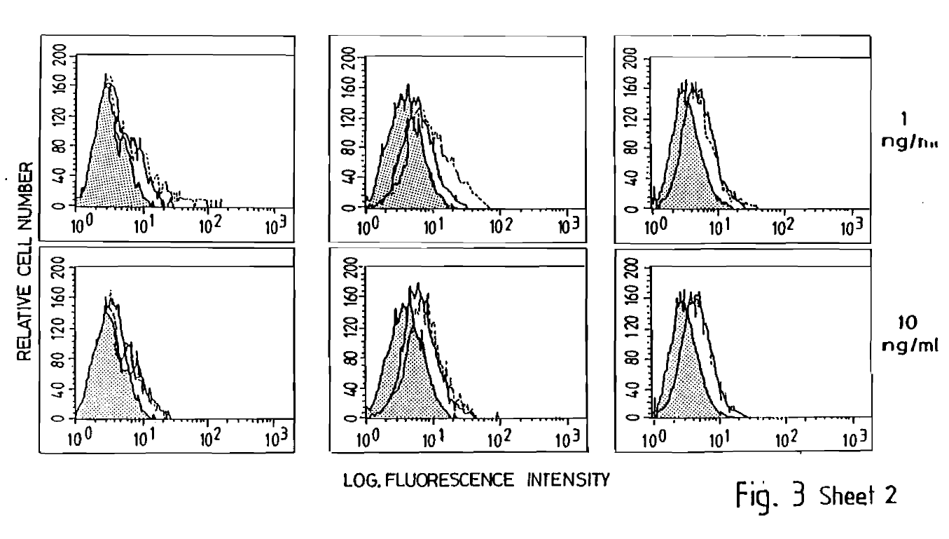

The selected population of pLAV12 transfectants (JLAV12S) was then cloned by limiting dilution and two subclones expressing the chTCR at low (2E11) and intermediate (IF5) levels (FIG. 3) were selected for further study.

Regulation of the Chimeric scFv-TCRζ Gene Expression.

To determine whether the expression of the chTCR could be suppressed by tetracyclines, JLAV12S and JN3S cells (5×10⁵/ml) were incubated for 48 hours in the presence of 1 μg/ml of tetracycline (Tet) or its analogue doxycycline (Dox). At this concentration most of surface chTCRs (90%) were down-regulated in JLAV12S cells, but were not affected in JN3S cells (FIG. 2A). Also, surface staining with anti-CD3ε mAbs demonstrated that the amount of TCR/CD3 complex remained constant in both cell populations (not shown). No changes in cell viability were observed at this concentration, assayed using trypsan blue staining (data not shown).

To study the time-course of inactivation of gene expression, JLAV12S cells were analysed at different times after addition of the antibiotics at 1 μg/ml (FIG. 2B). A slight reduction was observed within 8 hours of exposure to the drugs and maximum repression was achieved within 24 hours, when the expression of the chTCR fell to less than 10 percent of the level observed in the absence of tetracyclines at the same time point (FIG. 2B). Similar results were observed in the 1E5 and 2E11 clones where the level of chTCR was reduced to about 10% (1E5) and 20% (2E11) of its maximal expression (FIG. 3). The time-course of gene repression was very similar in response to both antibiotics (Tet or Dox) in all analysed populations. It is important to note that the percentage of cells in which tetracyclines did not down-regulate the expression of chTCRs was<0.5%, not affecting the overall regulation in the whole population of JLAV12S cells (FIG. 2A).

A dose-response curve of gene repression was determined using different concentrations of Tet or Dox. After 48 hours of treatment, the cells were harvested and the expression of the chTCR was studied by FACS analysis. Typical results are shown in FIG. 3.

Stably transfected uncloned (JLAV12S, left hand column) and cloned (1F5, middle column; and 2E11, right hand column) Jurkat cell populations were cultured for 48 hours in the presence of different concentrations (0 ng/ml top row, 0.1 ng/ml second row, 1 ng/ml third row, and 10 ng/ml bottom row) of Tet (broken line) or Dox (solid line) and the surface expression of scFv-TCR ζ molecules was examined. Negative controls (FITC-conjugated goat antisera to mouse IgG) are overlaid (filled curve). The fluorescence channel number is plotted along the x axis, and the y axis represents the relative cell number.

Referring to FIG. 3, in both clonal populations (1E5 and 2E11) the expression of chTCRs was maximally repressed at 100 pg/ml (0.1 ng/ml) of Dox, with no further increment in the level of repression at higher concentrations. Partial activity of the TRP was observed in the concentration range of 1 pg/ml to 100 pg/ml of Dox (data not shown). In JLAV12S cells maximal repression was observed at Dox concentrations greater than or equal to 1 ng/ml. For tetracycline maximal repression occurred, in all the analysed cell lines, at concentrations greater than or equal to 10 ng/ml. The induction of the chTCR gene was only partially repressed at tetracycline concentrations of 100 pg/ml to 1 ng/ml.

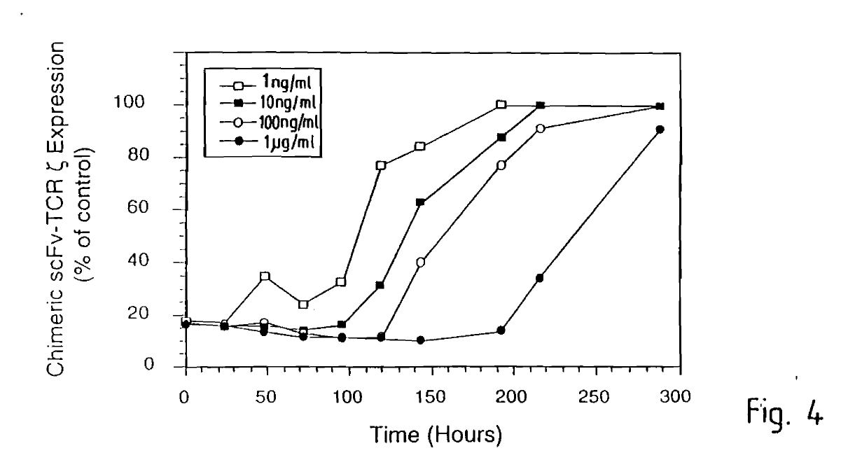

To study the kinetics of recovery of TRP-driven gene expression after withdrawal of tetracyclines, stable transfected uncloned JLAV12S cells were cultured for 48 hours in the presence of different concentrations of Dox (1 ng/ml to 1 μg/ml). After three washes the cells were incubated (5×10⁵/ml) in tetracycline-free CM in new plates and the surface expression of chTCRs was examined every 24-48 hours after staining with FITC-conjugated goat antisera to mouse λ light chain. The results are shown in FIG. 4. Similar experiments were also performed with 1F5 cells (data not shown).

100% values correspond to the median fluorescence of control untreated cells after staining with FITC-conjugated goat antisera to mouse light chain. The values are the percentage of chimeric TCRζ molecules expressed on tetracycline treated cells from each cell population as compared with the amount of chimeric TCRζ molecules on control untreated cells (as 100%). Referring to FIG. 4, after treating the cells with 1 μg/ml of Dox (filled circles), recovery of chTCR expression was not apparent at 192 hours after removal of the drug and was first detected on the cell surface after 216 hours, with fill recovery of expression after 288 hours. In contrast, the TRP remained repressed for only 24 hours after drug removal, when cells were pretreated with 1 μg/ml Tet, with maximal levels of chTCR expression being reached after 72 hours (data not shown). Treating the cells with lower concentrations of Tet (not shown) or Dox (1 ng/ml—open squares, 10 ng/ml—filled squares, 100 ng/ml—open circles) resulted in earlier recovery of the activity of the TRP.

### EXAMPLE 2

### Functional Study

It has previously been shown that the chTCR employed in this study is able to mediate specific recognition of its cognate antigen (NIP conjugated to BSA), soluble or plastic immobilized, resulting in the production of IL-2 by the transfected T cells (12). In the current study the inventors found consistently that stimulation of chTCR expressing cells (JN3S, JLAV12S, IF5 and 2E11) with plastic immobilized NIP₁₀-BSA conjugates induced IL-2 secretion (data not shown). The level of IL-2 production varied between different transfectant cell populations, but in general it was similar to that observed in the same cell population in response to standardised stimulation with anti-CD3ε mAb immobilized in microtiter wells (not shown).

Given that high concentrations of tetracycline have been shown to interfere with the process of T cell activation (17), the inventors studied the effects of increasing concentrations of Tet and Dox on the anti-CD3ε induced IL-2 secretion of Jurkat cells. IL-2 secretion was unaffected by 100 ng/ml or lower concentrations of doxycycline but was inhibited by 25% at a doxycycline concentration of 1 μg/ml (not shown). Tetracycline at concentrations lower than or equal to 1 μg/ml did not influence the anti-CD3ε induced IL-2 secretion, which was similar to that observed in untreated cells (not shown). These results indicate that is possible to induce maximal TRP repression at concentrations of doxycycline more than 1000-fold lower than the threshold at which immunomodulating effects on human T cells are first manifest.

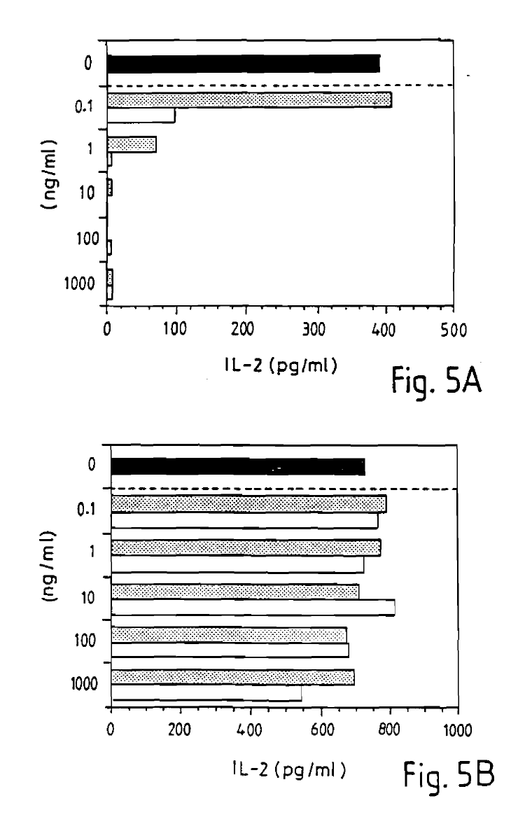

The inventors next determined whether tetracycline-mediated suppression of the chTCR could induce a reversible state of antigen unresponsiveness in the transfected Jurkat cells. JLAV12S and JN3S cells were preincubated for 48 hours in increasing concentrations of Dox and Tet and then stimulated with immobilized NIP-BSA, following which their IL-2 production was measured. In situations of full TRP repression JLAV12S cells down-regulated 90% of surface chTCRs (see FIG. 3), and were completely unresponsive to stimulation with iNIP10-BSA conjugates (see FIG. 5A).

FIGS. 5A and 5B are bar charts showing IL-2 production (in pg/ml) by transfected JLAV12S (5A) or JN3S (5B) cells stimulated with iNIP10-BSA in the absence or presence of Tet or Dox. Cells were preincubated for a 48 hours period in the absence or presence of the drugs (at the indicated concentration in ng/ml), washed and stimulated (10⁵/well) with plastic immobilised NIP10-BSA conjugates (50 μg/ml) in fresh CM alone (solid bar) or in the presence of Tet (shaded bars) or Dox (open bars) at the indicated concentrations. One of two similar experiments is shown.

Down-regulation of about 75% of chTCRs was associated with low IL-2 production, whereas no inhibitory effect was observed when the level of chTCR expression was about 50% of that observed in absence of tetracycline (FIG. 5A). These results indicate that the number of surface chTCR molecules expressed by JLAV12S cells in situations of full TRP repression is not enough to reach the activation threshold required for optimal T cell function. In contrast, when JN3S cells were stimulated with iNIP10-BSA conjugates, there was no inhibitory effect of tetracycline and of doxycycline at a concentration of less than 1 μg/ml (FIG. 5B).

This example shows the feasibility of using regulated expression of leukocyte-activating molecules on the surface of leukocytes to render the leukocyte differentially sensitive to different densities of leukocyte-stimulating molecules (such as antigens) present on the surface of a target cell.

Discussion

The inventors have used a single vector containing all of the components of the TRS for the pharmacological regulation of a foreign gene expressed in a human T cell line. The TRS comprises the tTA gene, usually driven by a constitutive promoter, and a gene of interest immediately downstream of the tTA-responsive promoter (9). To facilitate the application of the TRS to T cells, the inventors constructed a self-contained plasmid vector encoding both components of the TRS, as well as a hygromycin selectable marker gene under the control of a constitutive promoter. The new vector overcomes the efficiency losses inherent in co-transfection with the original two-plasmid based TRS system (9) and ensures the integration of equal copy numbers of the tTA and reporter gene units in a direct cis-configuration at the same chromosome locus.

Using this stable expression system as a model for integrated gene therapy approaches the inventors have demonstrated that a scFv-TCRζ chimeric molecule can be functionally expressed in a human T cell line, and that its expression can be pharmacologically down-modulated leading to loss of responsiveness to the targeted antigen in the genetically modified T cells. In the absence of tetracyclines the level of expression of the chTCR was comparable to that observed when the chimeric gene was driven by the strong CMV IE enhancer/promoter. Efficient tetracycline-dependent suppression of gene expression was observed in all the studied T cell transfectants. Depending on the dose and analogue employed, the expression of chTCRs could be repressed to a greater or lesser extent. This indicates that the TRS is applicable to T cells, achieving functional levels of transactivator expression without any evident toxic squelching effects of the VP16 domain.

Variable potencies have been demonstrated for different tetracycline analogues in previous studies on the TRS with doxycycline exhibiting 100-fold greater potency than tetracycline (18). In our system maximum repression occurred at a concentration of 10 ng/ml tetracycline whereas doxycycline caused full repression of the TRP over the range of 100 pg/ml to 1 ng/ml.

The time-course for the decline in chTCR expression upon exposure to Tet or Dox was short and suggests that, like wild-type TCRζ chains the chimeric TCRζ chains exhibit rapid turnover (19). In this regard it is interesting to note that the turnover of TCRζ chains in Jurkat cells is similar to that in primary T cells (19). In contrast to the rapid suppression of gene expression, its recovery after removal of doxycycline was much slower. The delay before the commencement of recovery of chTCR gene expression varied in direct proportion to the concentration of doxycycline that was used for repression. This sustained repression and relatively slow recovery of gene expression which occurs upon removal of tetracycline analogues has been observed in other cell types and could be of interest in the development of new T cell immunotherapy approaches and new immune evasion strategies.

Expression of the chimeric TCR conferred responsiveness to NIP-BSA conjugates, as evidenced by IL-2 secretion upon exposure to the antigen. Doxycycline was shown to completely abrogate this antigen responsiveness at concentrations that had no other immunomodulating effects on human T cells, and well below the tissue concentrations that are achieved clinically when doxycycline is used to treat infection (20). Thus, these results show that the use of TRS vectors can provide the means to render reinfused gene-modified T cells unresponsive to their targeted antigen if they cause autoimmune disorders. Likewise, by treating engineered T cells with doxycycline before their administration, it should be possible to switch off expression of the transgene for a predetermined period of time, thereby limiting collateral damage to normal tissues that may express low levels of the targeted antigen.

Interestingly, some residual expression of the chTCR in Tet or Dox treated Jurkat T cells was always detectable by FACS analysis, even when suppression was sufficient to completely abrogate responsiveness to the targeted antigen. This indicates that the number of chTCR molecules expressed in the “off” state is not enough to achieve efficient T cell activation (21), even with the experimental system used, where the cells were exposed to high concentrations of multivalent antigen. The ability to regulate easily the expression of TCR molecules at defined levels could be relevant to studies of the stoichiometry of the T cell activation process. On a therapeutic level, this points to the possibility of rendering T cells differentially sensitive to different surface densities of the antigen on target cell membranes.

Complete suppression of transgene expression was not fully achieved in the current study. The characteristics of the employed vector and/or cell type-specific factors may account for the level of transcription observed in the uninduced state (22, 23). In addition, the potential immunogenicity of the tTA protein must be taken into consideration. In the vector that we have employed the transactivator is driven by a constitutive promoter ensuring constant expression independent of the presence of tetracycline, and may therefore possibly elicit an immune response against the genetically modified T cells, even in the repressed state. However, this is not the case for a newer generation of enhancerless tetracycline-responsive vectors, in which tetracycline prevents the tTA protein from binding to the TRP, thereby suppressing its own expression as well as the expression of the reporter gene by way of an autoregulatory circuit (18, 24). This arrangement gives enhanced suppression of transgene expression (to negligible levels in preliminary experiments) and ensures that the abundance of the tTA protein is greatly reduced in the fully repressed state (24).

In summary we have demonstrated the use of a single tetracycline-responsive vector to achieve tetracycline-suppressible expression of a chimeric TCR gene in Jurkat T cells and we have furthermore shown that this provides a convenient method for the pharmacological regulation of the responsiveness of the engineered T cells to their targeted antigen.

### REFERENCES

1. Mule et al., 1984 Science 225:1487.
2. Rosenberg et al., 1986 Science 233:1318.
3. Eshhar et al., 1993 Proc. Natl. Acad. Sci. USA. 90: 720.
4. Hwu et al., 1993 J. Exp. Med. 178: 361.
5. Stancovski et al. 1993 J. Immunol. 151: 6577.
6. Brocker et al. 1996 Eur. J. Immunol. 26:1770.
7. Pardoll 1994 Cancer. Curr. Opin. Immunol. 6:705.
8. Yarranton 1992 Curr. Opin. Biotechnol. 3:506.
9. Gossen & Bujard 1992 Proc. Natl. Acad. Sci. USA. 89:5547.
10. Gossen et al. 1993 Trends Biol. Sci. 18:471.
11. Spits et al. 1985 Eur. J. Immunol. 15:88.
12. Alvarez-Vallina & Hawkins 1996 Eur. J. Immunol. 26: 2304.
13. Patten et al. 1992 J. Immunol. 150:2281.
14. Alvarez-Vallina et al. 1993 J. Immunol. 150: 8.
15. Hawkins et al. 1992 J. Mol. Biol. 226: 889.
16. Weissman et al. 1988 Proc. Natl. Acad. Sci. USA. 85: 9709.
17. Kloppenburg et al. 1995 Clin. Exp. Immunol. 102:635-641.
18. Hofmann et al. 1996 Proc. Natl. Acad. Sci. USA. 93: 5185-5190.
19. Ono et al. 1995 Immunity. 2:639-644.
20. Houin et al. 1983 Br. J. Clin. Pharmac. 16:245.
21. Valitutti et al. 1995 Nature. 375:148.
22. Shockett & Schatz. 1996 Proc. Natl. Acad. Sci. USA. 93:5173.
23. Ackland-Berlund & Leib. 1995 BioTechniques. 18:196.
24. Shockett et al. 1995 Proc. Natl. Acad. Sci. USA. 92:6522.

### Other Embodiments

Other embodiments will be evident to those of skill in the art. It should be understood that the foregoing detailed description is provided for clarity only and is merely exemplary. The spirit and scope of the present invention are not limited to the above examples, but are encompassed by the following claims.

1-18. (canceled)

19. A method of regulating the expression of a nucleic acid sequence encoding a polypeptide which is immunogenic in a mammal, wherein said mammal has already made an immune response to said polypeptide, said method comprising:
(a) obtaining a cell comprising a vector comprising a drug-regulatable promoter operably linked to said nucleic acid sequence encoding said polypeptide;
(b) introducing said cell into said mammal; and
(c) increasing expression of said polypeptide as a result of altering the amount of regulatory drug to which said cell is exposed.

20. The method of claim 19, wherein said cell is a leukocyte.

21. The method of claim 19, wherein said cell is a B lymphocyte, T lymphocyte, monocyte, or macrophage.

22. The method of claim 19, wherein said immune response comprises circulating antibodies against said polypeptide.

23. The method of claim 19, wherein said drug-regulatable promoter is a tetracycline-regulatable promoter.

24. The method of claim 19, wherein said regulatory drug is tetracycline or a tetracycline analog.

25. The method of claim 19, wherein said increase in expression of said polypeptide occurs two or more days after said introducing step.

26. The method of claim 19, wherein said vector is a viral vector.
